(19) United States
(12) Patent Application Publication
(10) Pub. No.: US 2012/0108888 A1
(43) Pub. Date: May 3, 2012

(54) METHOD AND DEVICE FOR AROMATHERAPY

(76) Inventor: Donald SPECTOR, New York, NY (US)

(21) Appl. No.: 13/314,782

(22) Filed: Dec. 8, 2011

Related U.S. Application Data
(63) Continuation-in-part of application No. 12/535,448, filed on Aug. 4, 2009, now abandoned.
Application No. 12/535,448 is a division of application No. 12/040,208, filed on Feb. 29, 2008, now abandoned.
Application No. 12/040,208 is a continuation of application No. 10/322,946, filed on Dec. 18, 2002, now abandoned.
Application No. 10/322,946 is a continuation-in-part of application No. 09/797,335, filed on Mar. 1, 2001, now Pat. No. 6,520,826.
(60) Provisional application No. 60/271,397, filed on Feb. 27, 2001.

(51) Int. Cl. A61M 21/02

(52) U.S. Cl. 600/27

(57) ABSTRACT

A fragrance dispensing device has a shape correlated with the fragrance being dispensed and when affixed with a nozzle can be inserted into a person's nose to provide aromatherapy or other herbal medications. The fragrance dispensing has a hollow body having a shape of a character and having a slit valve or a one-way valve disposed within the wall of the hollow body. A scented cartridge is inserted into the hollow body through the valve or some other resealable opening in the hollow body. The amount of fragrance dispensed is based, in part, upon the volume of the hollow body and the partial pressure of the fragrance within the cartridge. Where additional scent is desired, the hollow body can be repeatedly compressed to discharge additional discrete charges of fragrance into the environment.

### CROSS REFERENCE TO RELATED APPLICATIONS

This application is a continuation in part of U.S. patent application Ser. No. 12/535,448, filed on Aug. 4, 2009, which is a divisional application of U.S. patent application Ser. No. 12/040,208, filed Feb. 29, 2008, now abandoned, incorporated herein by reference in its entirety, which is a continuation of U.S. patent application Ser. No. 10/322,946, filed Dec. 18, 2002, now abandoned, which is a continuation-in-part of U.S. patent application Ser. No. 09/797,335, filed Mar. 1, 2001, now U.S. Pat. No. 6,520,826 issued Feb. 18, 2003, which in turn is based on Provisional Patent Application Ser. No. 60/271,397, entitled Plush Fragrancing System, filed Feb. 27, 2001.

### BACKGROUND OF THE INVENTION

1. Field of the Invention

This invention relates to fragrance dispensing devices. More specifically, this invention relates to a fragrance dispensing device wherein a discrete charge of scented air can be displaced from within a resilient deformable chamber located within the device. The discrete charge is contacted with a core material within the device, and, thereafter, can be dispensed into the surrounding.

2. The Prior Art

The process of dispensing of perfumes, scented fluids and solid air fresheners from various contrivances is well-known. Generally, such processes involve aspirating a finite charge of scented liquid from a reservoir, or alternatively, allowing a scented liquid or solid to passively evaporate/sublime into the atmosphere at a known or controlled rate. The following patents are representative of devices which utilize one or more of the above processes for dispensing a scent into the ambient environment.

U.S. Pat. No. 6,089,947 (to Green, issued Jul. 18, 2000) discloses an air freshener representing a figure, such as an animal, cartoon character, or other form having a body and a mouth. The body of the Green air freshener is made of an air-permeable material (e.g. plush fabric) and forms an internal cavity within the body. A mouth leads through the body and into the internal cavity so that a scented pellet can be inserted through the mouth and retained within the internal body cavity. The scented pellet then releases a permeating scent to the ambient or surrounding air. Once spent, fresh pellets can be added through the mouth of the figure.

U.S. Pat. No. 4,889,284 (to Spector, issued Dec. 26, 1989) discloses a rechargeable air freshener in a figurative form that includes a body and a head section, and an outer casing of permeable fabric material whose contours define all sections of the figure. The interior of the casing and all sections thereof are stuffed with a compressible core of absorbent material having good wicking properties, the core rendering the figure soft and squeezable. Occupying an internal cavity in the core, which extends from the body to the head section thereof, is a fragrance dispenser comprising a cylindrical can filled with liquid fragrance, a depressible stern projecting from one end of the can terminating in an actuator head provided with a spray nozzle. The can is so placed in the cavity that the actuator head lies just below the scalp region of the head section of the casing. When, therefore, the user presses the scalp region of the figure, this actuates the dispenser to spray a mist of liquid fragrance onto the core material within the head section, the fragrance being absorbed thereby and thereafter wicked throughout the core. The aromatic vapor is volatilized from the outer surface of the core, passes through the permeable casing and, thus, released into the atmosphere.

As is evident from the above discussion of the representative prior art, each of the foregoing fragrance dispensers require either the periodic insertion of a fragrance releasing sachet or pellet, or alternatively, the replenishment of a reservoir containing a scented liquid. In the case of the Green device discussed above, the scent is continuously released into the atmosphere until exhausted and replenished. Thus, the amount of scent present in ambient environment is independent of any control that can be exercised by the user. In the case of the Spector device, This fragrance dispensing system combines a traditional scent aspiration system with the modulating effect of the plush figure, to both limit the amount of scent that is dispensed into the atmosphere at one time and yet extend the effect thereof through an intermediate medium which comprises core material of the plush figure.

In the Spector device, the recharging of the fluid reservoir containing the liquid fragrance is more cumbersome than in Green, notwithstanding its apparent advantages. Accordingly, there continues to exist a need for a fragrance dispensing figure having the favorable attributes of Spector device, with the ease of replenishment of the fragrance source of the Green device.

It is the object of this invention to remedy the above as well as related deficiencies in the prior art.

More specifically, it is the principle object of this invention to provide an improved fragrance dispensing device and method including means for dispensing a discrete charge of fragrance from a reservoir that can be readily replaced or replenished.

It is another object of the invention to provide a fragrance dispensing device in which the device has a shape that correlates with the fragrance being dispensed.

It is another object of this invention to provide an improved fragrance dispensing device for use in aromatherapy wherein a discrete charge of fragrance is dispensed from a source within the device by simply compressing a flexible container within the device.

It is still yet another object of this invention to provide an improved fragrance dispensing device wherein a discrete charge of fragrance is dispensed from a scented cartridge having concentrated fragrance adsorbed upon a solid carrier material. A further object is to provide a dispensing device which contains a nozzle or tubular extension which can be inserted into a person's nose to provide aromatherapy. A still further object is to provide a method for administering aromatherapy to a person employing the dispensing device of the present invention. These and other objects will readily become apparent to those skilled in the art in light of the teachings disclosed herein.

### SUMMARY OF THE INVENTION

The above and related objects are achieved by providing a fragrance dispensing system and device comprising a compressible, resilient hollow body having a cartridge with a concentrated fragrance adsorbed on a solid, porous carrier. In a preferred embodiment of this invention, the hollow body can have porous walls and have a slit or valve. The device is also fitted with a nozzle or tubular extension having a one-way valve through which the fragrance can be dispensed in a specific direction, such as into a person's nasal passage. The cartridge is inserted into the hollow body through the valve or some other resealable opening in the hollow body. Once the sphere is charged with the fragrance cartridge, it can be compressed so as to force a discrete charge of scented air from within the hollow body, through the nozzle into its surrounding environment. The amount of fragrance dispensed from within the hollow body is based, in part, upon the volume of the hollow body and the partial pressure of the fragrance within the cartridge. Thus, if the fragrance is highly volatile, more fragrance will be present in the discrete charge released from the hollow body. Where additional scent is desired, the hollow body can be repeatedly compressed to discharge additional discrete charges of fragrance into its immediate environment.

In order to prolong the effect of such dispenser, the hollow body is preferably embedded within a compressible container or device. The shape of the hollow body will, thus, conform in overall dimension with its intended situs within the container. The container and/or the hollow body preferably have a shape corresponding to the fragrance being dispensed. For example, if the fragrance is fruit flavored, the hollow body and container could be in the shape of the particular fruit, such as an apple or strawberry. If the fragrance is a mixture of herbal scents which had a particular effect on people, the container and hollow body could be shaped to reflect this effect. For example, if the fragrance is supposed to induce a calming effect, the hollow body or container could be shaped like a sleeping animal.

The advantages of the fragrance dispensing system of this invention include the ability to recharge the system by simply replacing the fragrance dispensing cartridge in the hollow body, or by totally replacing the entire hollow body with a new one having a fresh fragrance dispensing cartridge. The system also has the advantage of isolating the source of the fragrance from direct contact with fabrics and similar materials that can be wetted and/or stained and/or discolored by contact with the fragrance. Hence, when used for aromatherapy, only an essentially dry vapor is dispensed from the system. Moreover, the replenishment of the system does not involve, nor require, the use of liquids which are cumbersome and difficult to handle, particularly in the context of re-filling of a tiny reservoir.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 depicts a compressible hollow body of this invention having a resilient compressible chamber suitable for placement within a container or device.

FIG. 2 depicts an enlarged view of a cartridge suitable for use in a fragrance dispensing device of this invention;

FIG. 3 depicts one embodiment of a dispensing device useful for administering aromatherapy; and

FIG. 4 depicts another embodiment of a dispensing device according to the invention.

### DETAILED DESCRIPTION OF THE PREFERRED EMBODIMENT

The fragrance dispensing system and device of this invention comprises a resilient, compressible hollow body having a cartridge, or a similar dispenser, having a concentrated fragrance adsorbed onto a porous solid support (e.g. sponge). The compressible hollow body, suitable for use in the system of this invention can be in the form of a sphere or globe, or can be in a shape of a character or other shape that corresponds with the fragrance being dispensed. The compressible hollow body defines a chamber having a scented dispenser disposed therein. The hollow body is further provided with a simple valve which is normally in the closed or sealed position until the hollow body is compressed. The exertion of compressive forces on the hollow body cause displacement in the wall of the globe and thereby the opening of the valve. When a discrete charge of fragrance is present within the chamber, such compression cause discharge of the discrete charge from within the internal chamber defined by the hollow body. The hollow body is preferably provided with an additional one way valve to permit air to enter the internal chamber of the hollow body upon the release of the compressive pressure, thus, re-filling the globe with air. In a preferred embodiments of this invention, air entering the internal chamber of the hollow body is caused to impinge or flow over the dispenser contained therein, and, thus, is combined with fragrance from the dispenser. This process can be repeated, and additional scented air discharged from the internal chamber of the hollow body. It is noted and emphasized that only so much of the fragrance as can be accommodated by the volume of air within the internal chamber is discharged with each compression of the hollow body. This configuration, thus, permits precise control over the amount of scent that is dispensed from the dispenser and greatly extends the useful life of the dispenser, while at the same time preventing overwhelming the surroundings with scented air particularly when used for aromatherapy. In a preferred embodiment the dispenser is in the form of a cartridge as described below.

In order to further control this process, the fragrance dispensing system is included within a container. In the plush figure described in commonly assigned U.S. Pat. No. 4,889,284 (to Spector, issued Dec. 26, 1989), which is herein incorporated by reference in its entirety, the scent is dispensed by aspiration of a liquid spray from a fluid reservoir within the container onto a core material in the body of the container. The aspirated liquid spray is absorbed onto the core material which transports the liquid, by capillary action, throughout the core. The core material, to be effective in this environment is preferably comprised of a synthetic, non-porous fiber, that is essentially inert and hypoallergenic. In the context of this invention, the core material provides a comparable function to a passive scent dispensing medium—namely, its provides a high surface area medium for effecting diffusion and exchange of a defined volume of air and fragrance between the core and the environment external to the container. As is appreciated, this dispensing of a discrete charge of fragrance from the fragrance dispensing cartridge of the compressible hollow body within the container provides not only precise control over the amount of fragrance that is contained within each charge of scented air, but also isolates the fragrance dispensing cartridge so as to prevent passive diffusion of fragrance from the cartridge. Thus, the unique fragrance dispensing system of this invention not only extends the useful life of the cartridge, but also prevents the release and/or dispensing of an excessive amount of fragrance into the ambient environment or when used for aromatherapy. The core material of the plush figure also cooperates in this regard, by retaining a residual scent that can gradually be evolved where the container itself is in an environment that causes air flow over the exterior surface of the figure. Thus, long after the discrete charge of the fragrance has been dispensed from the compressible hollow body, the core provides an intermediate mechanism for continuous, metered release of fragrance.

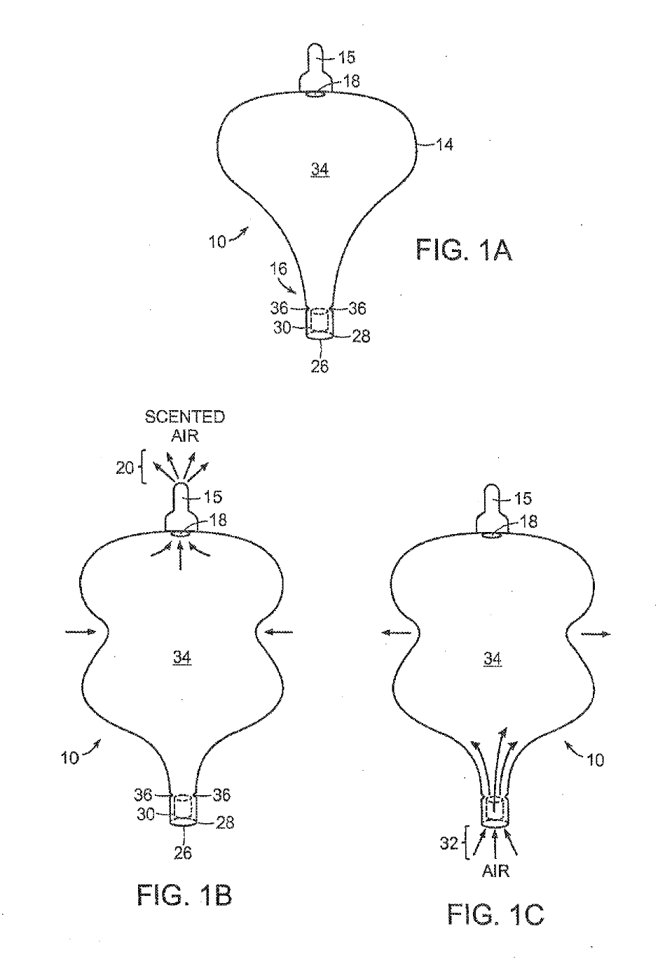

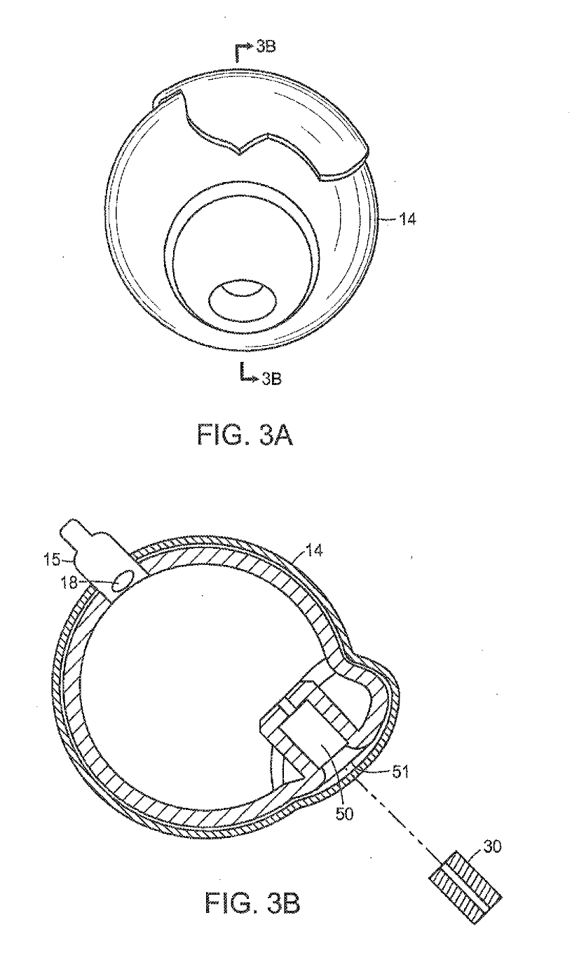

In the embodiments of the invention illustrated in FIGS. 1A, 1B and 1C, a hollow compressible body (10) can be in the form of a pear shaped form having an essentially bulbous head (14), a nozzle or tubular extension (15) and an elongate tube (16), each of nozzle (15) and tube (16) having a one way valve (18, 26) positioned at each end thereof. As more completely illustrated in FIG. 1A, the hollow compressible body (10) includes a one way discharge valve (18) on the proximal end in nozzle (15) thereof. Discharge valve 18 leads into a first elongate tube or nozzle (15) which is configured to fit into a person's nose for administration of nasal therapy. Tube (15) can contain a removal cap not shown. As shown in FIG. 1B, upon compression of the compressible body, a discrete charge of fragrance (20) is dispensed from nozzle (15). The distal end of the elongate tube (16) includes a one-way inlet valve (26) and a removal cap (28). The ability to remove the cap from the distal end of the tube allows for access to the chamber (30) in such tube and thereby the insertion and replacement of an in-line cartridge, or similar dispenser, within the hollow cavity of the tube (16). The one-way inlet valve (26) in the removable cap (28) allows for air to enter the elongate tube and thereby pass through the cartridge within the tube and thereafter into the hollow cavity at the proximal end of the compressible body. Accordingly, when the proximal end (14) of the compressible body is compressed, as illustrated in FIG. 3B, scented air (20) contained therein is discharged into the core of the container not shown. Upon release of such compression, as illustrated in FIG. 3C, a negative pressure is created within the chamber (34). This negative pressure causes fresh air (32) to be drawn into the distal end of the tube (16) through the one-way inlet valve (26) located in the cap (28) on the distal end of the tube. As air (32) flows into the tube (16), it is channeled into the cartridge (30), where it impinges upon a solid support having the concentrated fragrance. Such contact causes a finite amount of fragrance to combine with the air and fill the chamber (34) located in the proximal end (14) of the compressible body (10).

In a further embodiment of the present invention, the hollow body can contain an inert core material, not shown, which is preferably comprised of a synthetic, non-porous fiber that is essentially inert and hypoallergenic. The core material promotes a high surface area and functions as a passive scent dispensing medium for effecting diffusion and exchange of a defined balance of air and fragrance between the core and the external environment.

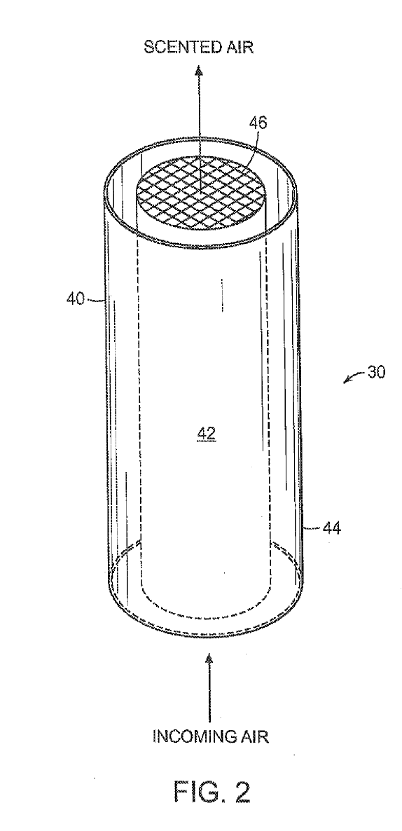

The fragrance dispensing cartridge, or similar dispenser (30) can take many forms and include one or more combinations of scents. In the embodiment of the invention illustrated in FIG. 2, the cartridge comprises a cylindrical housing (40) having a central channel (42) defined by a porous material (44), in the form or a plastic foam or sponge like material. The foam is essentially inert relative to the concentrated fragrance that is adsorbed onto its surface and within the bibulous matrix thereof. The porous material (44) is retained within the channel (42) of the cartridge (30) by a screen or mesh (46), so as to permit incoming air to impinge upon such porous material. Fragrance dispensing cartridges of the type suitable for use in this invention are disclosed in U.S. Pat. No. 4,523,870, which is herein incorporated by reference in its entirety. The porous material can also include other agents to stabilize the fragrance concentrate, or otherwise control its release from the medium. The overall dimensions of the fragrance dispensing cartridge are dictated by the shape of the cavity in which it is to be placed. For example, where the fragrance dispensing cartridge is used in the configuration of the device illustrated in the figures accompanying this application, it is preferably in the form of a cylinder having an opening on each end thereof. Thus, it can be inserted into the distal end of the elongate tube, and positioned in-line with the direction for passage of air from one the distal end of the tube to the proximal end of the compressible chamber. When air is directed through the cartridge it contacts and the carrier surface of the cartridge having absorbed fragrance concentrate. In this process, fragrance concentrate is once again combined with the air stream and flows into the proximal end of the compressible body, so as replenish the device with another “discrete charge” of scented air.

In order to retain the fragrance dispensing cartridge in position within the elongate tube (16), retainer means (36) can be provided either within the distal end of the tube (16), or on the interior surface of the removable cap (28), or on both. More specifically, the interior of the distal end of the elongate tube (16) is also provided with a constriction or restriction means (36) to secure the cartridge (30), in-line, in the distal end of such tube. Thus, upon compression of the proximal end (14) of the compressible body (10), a discrete volume of scented air (20) is discharged from the interior chamber (34) thereof through the one-way dispensing valve (18) and out nozzle (15) of the compressible body (10). Upon relaxation of the compressive forces upon the compressible body (10), the compressible chamber (34) returns to its normal, original position. In the course of this sequence, a negative pressure is created within the compressible chamber, so as to draw fresh air (32) from the environment, external to the plush figure, through the one-way inlet valve (26) in the removal cap (28) on the distal end of the tube (16). The air (32) entering through the one-way inlet valve (26) in the cap is directed, through the in-line, cartridge (30), wherein it causes concentrated fragrance from the cartridge to mix with the in-coming fresh air, so as to once again charge the compressible chamber (34) with a discrete amount of fragrance. This process can be repeated over and over again until the concentrated fragrance in the cartridge is consumer/exhausted. The end-cap (28) on the distal end of the elongated tube (16) can then be removed, the exhausted fragrance dispensing cartridge replace with a new in-line fragrance dispensing cartridge. The ability to replace the cartridge provides versatility in the number of different fragrances that can be used with the same container. Access to the removal cap (26) is through a cover (not shown) in the container (12), or in any other convenient location. Thus, the container cover is opened and the cap removed from the distal end of the tube. The spent cartridge is thereafter removed from the opened tube and new cartridge inserted. The cap is then replaced, and the cover of the container closed.

Another embodiment of a fragrance dispenser is shown in FIG. 3. In FIG. 3, the compressible body (14) is housed in a container (52) in the shape of a ball with nozzle or tube (15) and valve (18). Cartridge (30) fits into recess (50) and secured with outer plug (51).

Additional modifications to the preferred embodiments of the invention include the placement of the hollow compressible body in a container which can have various configurations and access means for nozzle (15) and recess (50). The form or shape of the hollow compressible body would, where practical, provide access to the fragrance dispensing cartridge to removal or replacement. As noted above, it may be cost effective or desirable to simple replace the entire hollow compressible body; and, in such instance, the container would include means for access and removal of the entire hollow compressible body.

It is known that the odors or scent of certain perfumes, spices, gums, wines, flowers, herbs and the like, have a pleasant and soothing effect on the senses and in many instances provides a feeling of well being. Thus, aromatherapy is currently finding use as a means of providing a temporary and soothing sensation by inhaling certain vapors in essentially a dry form. The device of the present invention is therefore particularly useful for aromatherapy treatment through the nasal passage which avoids the use of a liquid carrier. The “dry” spray can hold a variety of fragrances, such as lavender and the like, that are used in aromatherapy. Herbal products having a scent or therapeutic value can also be administered by nasal therapy using the device of this invention. The nozzle extension is merely inserted close to or part way into the nasal passage and the container squeezed to dispense a particular scent or herbal medication in an essentially dry form. The user of the device can control the concentrations of aroma or scent by the frequency in which the device is pressed or squeezed. The user can, of course, dispense and smell a small amount of scent or fragrance before inserting the nozzle mean or into his or her nose to determine whether the concentration is of sufficient strength and desirability.

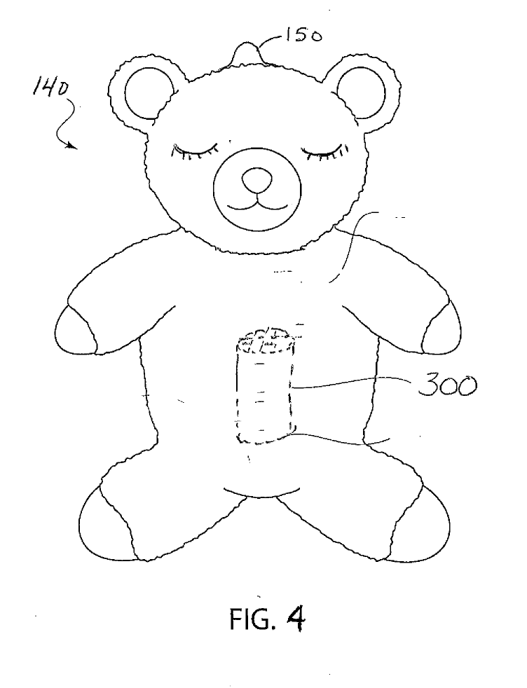

An alternative embodiment of the invention is shown in FIG. 4. Here, compressible body 140 is formed in the shape of a character, which can relate to the fragrance being dispensed. When the fragrance is used for aromatherapy, the compressible body 140 can indicate to the user the benefits of the fragrance. In this case, compressible body 140 is in the shape of a sleeping bear with a nozzle 150 on top, and fragrance cartridge 300 contains herbs that induce relaxation or sleep. Squeezing body 140 releases the fragrance from cartridge 300 through nozzle 150. Body 140 can be made of any suitable material, such as plastic or rubber. Body 140 operates with the same features as the other embodiments discussed above, but has a shape that is particular to the fragrance being dispensed. Body 140 could also be enclosed in a container (not shown), which can have the same or different shape from body 140, and could be made out of any type of porous or non-porous material, such as fabric or plastic.

Accordingly, while only a few embodiments of the present invention have been shown and described, it is obvious that many changes and modifications may be made thereunto without departing from the spirit and scope of the invention.

What is claimed is:

1. A method for administering aromatherapy to a person comprising:
providing a compressible hollow body having an opening in a wall of the hollow body to permit passage of air into a chamber of the body having fragrance concentrate absorbed on a solid porous medium within the chamber and having at least one valve with an opening through a nozzle to permit passage of a fluid vapor from the chamber and nozzle;
placing the nozzle into or near a nasal passage of said person; and
compressing said body to open the valve to dispense at least one charge of a fluid vapor having a fragrance into said nasal passage,
wherein the hollow body has a shape of a character or object and wherein the character or object correlates with the fragrance being dispensed.

2. The method of claim 1, wherein the fluid vapor is an herbal product.

3. The method of claim 1, wherein the fluid vapor is in essentially a dry form.

4. A method for administering aromatherapy to a person comprising:
providing a compressible, resilient hollow body with a fragrance concentrate absorbed on a porous material with the body the hollow body having a shape that correlates with the fragrance;
bringing a nozzle extension on the body toward a nose of the person, the nozzle extension having a valve with an opening;
compressing the hollow body to open the valve and dispense a discrete volume of scented air through the opening in the nozzle; and
inhaling at least a portion of the scented air.

5. The method of claim 4, wherein the scented air is in essentially a dry form.

6. The method of claim 4, further comprising inserting the nozzle extension into a nasal passage of the nose.

7. The method of claim 4, wherein inhaling at least a portion of the scented air generates a feeling of well being.

8. A device for administering aromatherapy to a person, comprising;
a compressible hollow body having an opening in a wall of the hollow body to permit passage of air into a chamber of the body;
a fragrance concentrate absorbed on a solid porous medium within the chamber; and
at least one valve with an opening through a nozzle to permit passage of a air or vapor from the chamber and nozzle;
wherein the compressible hollow body has a shape that is related to the fragrance concentrate, and wherein compressing the hollow body releases air or vapor containing fragrance from the fragrance concentrate out of the hollow body through the nozzle.

9. The device according to claim 8, wherein the compressible hollow body is in the shape of an animal.

10. The device according to claim 8, wherein the solid porous medium is encased in a cassette.

11. The device according to claim 8, wherein the compressible hollow body is made of plastic.

12. The device according to claim 8, wherein the solid porous medium is removable from the compressible hollow body.

13. The device according to claim 8, wherein the compressible hollow body is enclosed in a container, the container having the same shape as the compressible hollow body.
